(19) United States
(12) Patent Application Publication
(10) Pub. No.: US 2025/0302852 A1
(43) Pub. Date: Oct. 2, 2025

(54) COMPOSITIONS COMPRISING METHYLXANTHINES AND DISACCHARIDES DERIVED FROM ENZYMATIC CONVERSION OF SUCROSE

(71) Applicant: Fertis India PVT. Ltd., Hyderabad (IN)

(72) Inventors: Rahul Raju Kanumuru, Hyderabad (IN); Shaji George Kochumalayil, Hyderabad (IN); Ravikumar Suraneni, Hyderabad (IN); Murali Mohanarao Vana, Hyderabad (IN)

(73) Assignee: Fertis India PVT. Ltd., Hyderabad (IN)

(21) Appl. No.: 18/864,946

(22) PCT Filed: May 13, 2023
(86) PCT No.: PCT/IN2023/050452

§ 371 Date: Nov. 12, 2024

(30) Foreign Application Priority Data
May 17, 2022 (IN) 202141052783

(51) Int. Cl. A61K 31/7016; A61K 31/015; A61K 31/05; A61K 31/4985

(52) U.S. Cl. CPC A61K 31/7016; A61K 31/015; A61K 31/05; A61K 31/4985

(57) ABSTRACT

The present invention discloses compositions comprising methylxanthines in combination with disaccharides derived from enzymatic conversion of sucrose. More particularly, the present invention discloses compositions comprising methylxanthines and disaccharides derived from enzymatic conversion of sucrose selected from turanose, leucrose, trehalulose and isomaltulose or in combinations thereof.

### TECHNICAL FILED

The present invention relates to compositions comprising methylxanthines in combination with disaccharides derived from enzymatic conversion of sucrose. More particularly, the present invention relates to compositions comprising methylxanthines and disaccharides derived from enzymatic conversion of sucrose selected from turanose, leucrose, trehalulose and isomaltulose or in combinations thereof.

### BACKGROUND AND PRIOR ART

Methylated xanthines are part of foods, beverages, and dietary supplements and will be produced by a limited number of botanical species, including tea (Camellia sinensis L, C. assamica var. kucha (green tea)), coffee (e.g., C. arabica, C. liberica, C. dewevrei, C. abeokut), cacao (Theobroma cacao L.), Cola (e.g., C. nitida), Paullinia (e.g., P. cupana), and Ilex (e.g., I. paraguarensis).

Methylxanthines containing natural products are regularly used in food industry as dietary supplements, nutraceuticals or nootropics as a direct consequence of their ability to enhance long lasting and increased energy, mood, and focus, improve memory, cognitive enhancement, better mind muscle connection, performance enhancement, alertness and increase fat burning potential.

Pharmacological activity of methylxanthines at the cellular level are attributed to their ability in preventing the neurotransmitter adenosine from binding to receptors in the brain, inhibit phosphodiesterase enzyme, blocking dopamine transport proteins, modulation of GABA receptor action, and regulation of intracellular calcium levels.

Methylxanthines are also known to act as psycho stimulant, potent bronchodilator, enhances blood flow and prevent uric acid crystallization.

Although there are several dietary supplements containing methylxanthines are available, there is a need to develop a composition comprising methylxanthines in combination with disaccharides derived from enzymatic conversion of sucrose there by offers a blend of immediate-release, delayed-or extended-release, long lasting energy boosters which will result in improved mood, focus, memory, alertness, cognitive enhancement, better mind muscle connection, better performance, increase fat burning potential and information processing.

In addition to the above it is also important to mask the bitter taste of methylxanthines by developing an appropriate formulation.

Therefore, it is an objective of the present invention to provide compositions comprising methylxanthines in combination with disaccharides derived from enzymatic conversion of sucrose selected from turanose, leucrose, trehalulose and isomaltulose or mixtures thereof, so as to mask the bitter taste of methyl xanthines.

### SUMMARY OF THE INVENTION

Accordingly, the present invention provides compositions comprising methylxanthines in an amount of 0.01% to 20% w/w and disaccharide(s) derived from enzymatic conversion of sucrose in an amount of 10% to 90% w/w.

In an aspect, methylxanthines are selected from caffeine, theacrine and methyl liberine, paraxanthine, theobromine, theophylline either alone or combinations thereof in an amount of 0.01% to 20% w/w.

In another aspect, disaccharides derived from enzymatic conversion of sucrose are selected from the group comprising of turanose, leucrose, trehalulose and isomaltulose either alone or in combinations thereof in an amount of 10% to 90% w/w.

In an additional aspect, the invention provides compositions comprising individual methylxanthines selected from the group comprising of caffeine, theacrine, paraxanthine, theobromine, theophylline and methyl liberine in an amount of 0.01% to 20% w/w in combination with disaccharides derived from enzyme treated sucrose selected from the group comprising of turanose, leucrose, trehalulose and isomaltulose either alone or in combinations thereof in an amount of 10% to 90% w/w.

In another aspect, the composition of the present invention may comprise nutraceutical supplements like Stilbenoids selected from Resveratrol and Pterostilbene present in an amount of 0% to 20% w/w.

In another aspect, the composition of the present invention may comprise suitable carriers or excipients or nutritional adjuvant(s) in an amount of 0.01% to 20% w/w.

In another aspect, the composition of the present invention may comprise nutraceutical excipients in an amount of 5-90% w/w.

In yet another aspect, the present invention provides a process for preparation of the composition comprising methylxanthines alone or combinations thereof along with disaccharides derived from enzyme treated sucrose comprising;
i. Adding methylxanthines individually or in combination in an amount of 0.01% to 20% w/w to an aqueous solution of enzymatically treated sucrose in an amount 90 to 10% w/w;
ii. Adding stilbenoids like pterostilbene, resveratrol in an amount 0 to 20% w/w to the solution of step (i);
iii. Adding nutraceutical excipients in an amount 5 to 90% w/w to the solution of step (ii); and
iv. Agitating the solution of step (iii) at room temperature to avoid heat-induced inversion of sucrose to obtain the desired composition.

In one of the aspects, the compositions of the present invention can be formulated into various forms of nutritional supplements, such as powders, tablets, capsules, gel dosage forms, aqueous formulations, containing caffeine, theacrine and methyl liberine together with various isomers of enzyme treated sucrose which may be used as stimulant of the central nervous system and metabolic stimulant.

### DETAILED DESCRIPTION OF THE INVENTION

The present invention will now be described in detail in its preferred and optional embodiments so that the various aspects therein can be more clearly understood and appreciated.

Accordingly, the present invention discloses compositions comprising methylxanthines in combination with disaccharide(s) derived from enzymatic conversion of sucrose, wherein the methylxanthines in an amount of 0.01% to 20% w/w and disaccharide(s) derived from enzymatic conversion of sucrose in an amount of 10% to 90% w/w.

In yet another embodiment, the methylxanthines are either in their pure form or as cocrystals or solvates wherein the conformer/solvates is selected from the group comprising of Phenols, Cinnamic Acids, Resveratrol, Pterostilbene, alcohols, organic acids etc.

According to the invention, the methylxanthines are selected from the group comprising of caffeine, theacrine and methylliberine, paraxanthine, theobromine, theophylline or combinations thereof in an amount of 0.01% to 20% w/w.

In another embodiment, the disaccharides derived from enzymatic conversion of sucrose are selected from the group comprising of turanose, leucrose, trehalulose and isomaltulose or combinations thereof in an amount of 10% to 90% w/w.

In yet another embodiment, the invention provides compositions comprising individual methylxanthines selected from the group comprising of caffeine, theacrine, paraxanthine, theobromine, theophylline and methyl liberine in an amount of 0.01 to 20% w/w in combination with disaccharides derived from enzyme treated sucrose selected from the group comprising of turanose, leucrose, trehalulose and isomaltulose either alone or in combinations thereof in an amount of 10% to 90% w/w.

In another embodiment, the composition may comprise nutraceutical supplements like stilbenoids selected from resveratrol and pterostilbene in an amount of 0% to 20% w/w.

In yet another embodiment, the composition may comprise suitable carriers or excipients or nutritional adjuvant(s) in an amount of 0.01% to 20% w/w.

In another embodiment, the carriers or excipients or nutritional adjuvant(s) are selected from the group comprising of but not limited to flavouring agents, colorants, viscosity modifiers, preservatives, chelating agents, antioxidants, surface modifiers and other nutritional adjuvants alone or combinations thereof.

In yet another embodiment, the composition may comprise nutraceutical excipients in an amount of 5-90% w/w.

In another embodiment, the nutraceutical excipients selected from the group comprising of binders, diluents, lubricants, stabilizers, solubilizers, sustained release polymers, coloring agents, taste enhancers and the like.

In yet another embodiment, the composition optionally comprises bitterness masking agents.

In another embodiment, the composition may be formulated into either oral solid, liquid or suspensions.

In another embodiment, the composition may be formulated for oral administration in various forms of nutritional supplements, such as powders, tablets, capsules, gel dosage forms or aqueous formulations.

In another embodiment, the composition is provided as aqueous composition.

In another embodiment, the present invention provides a process for preparation of the composition comprising methylxanthines alone or combinations thereof along with disaccharides derived from enzyme treated sucrose comprising;
i. Adding methylxanthines individually or in combination in an amount of 0.01% to 20% w/w to an aqueous solution of enzymatically treated sucrose in an amount 90 to 10% w/w;
ii. Adding stilbenoids like pterostilbene, resveratrol in an amount 0 to 20% w/w to the solution of step (i);
iii. Adding nutraceutical excipients in an amount 5-90% w/w to the solution of step (ii); and
iv. Agitating the solution of step (iii) at room temperature to avoid heat induced inversion of sucrose to obtain the desired composition.

In another embodiment, the process for preparation of the composition comprising methylxanthines alone or combinations thereof along with disaccharides derived from enzyme treated sucrose wherein the methylxanthines selected from the group comprising of caffeine, theacrine, methyl liberine, paraxanthine, theobromine and theophylline; disaccharides selected from the group comprising of turanose, leucrose, trehalulose and isomaltulose; suitable carriers or excipients or nutritional adjuvant(s) selected from the group comprising of flavoring agents, colorants, viscosity modifiers, preservatives, chelating agents, antioxidants, surface modifiers and other nutritional adjuvants; and nutraceutical excipients selected from the group comprising of binders, diluents, lubricants, stabilizers, solubilizers, sustained release polymers, coloring agents, taste enhancers and the like.

In another embodiment, the compositions of the present invention can be formulated into various forms of nutritional supplements, such as powders, tablets, capsules, gel dosage forms, aqueous formulations, containing caffeine, theacrine and methyl liberine together with various isomers of enzyme treated sucrose, useful as a stimulant of the central nervous system and metabolic stimulant.

In yet another embodiment, the present invention provides compositions useful to substantially enhance long lasting and increased energy, mood, and focus, improve memory, cognitive enhancement, increase, maintained extend locomotor activity, performance enhancement, alertness, increase fat burning potential, increase endurance, reduce depression, impart a beneficial hypoglycemic effect and alleviate sleep deprivation.

In another embodiment, the present invention provides compositions useful to substantially improve endurance, exercise performance, reduced hunger, and weight loss, and inflammation and pain reduction, prophylactic and curative treatment for neurological disease, learning performance, mood enhancement, alertness, uric acid reduction and management.

In yet another embodiment, the composition provides delayed or extended-release composition.

In another embodiment, the present invention provides a method of substantially stimulating the central nervous system and metabolic stimulant in a subject wherein the method comprises providing said subject with a composition comprising methylxanthines in combination with disaccharide(s) derived from enzymatic conversion of sucrose in nutraceutically effective amount.

In another embodiment, the present invention provides a method of substantially improving endurance, exercise performance, reduced hunger, and weight loss, and inflammation and pain reduction, prophylactic and curative treatment for neurological disease, learning performance, mood enhancement, alertness, uric acid reduction and management in a subject wherein the method comprises providing said subject with a composition comprising methylxanthines in combination with disaccharide(s) derived from enzymatic conversion of sucrose in nutraceutically effective amount.

In another embodiment, the present invention provides a method of substantially enhancing long lasting and increased energy, mood, and focus, improving memory, cognitive enhancement, increase, maintain extend locomotor activity, performance enhancement, alertness, increase fat burning potential, increase endurance, reduce depression, impart a beneficial hypoglycemic effect and alleviate sleep deprivation in a subject wherein the method comprises providing said subject with a composition comprising methylxanthines in combination with disaccharide(s) derived from enzymatic conversion of sucrose in nutraceutically effective amount.

In an embodiment, the present invention provides compositions suitable for oral administration to mammalian subjects, including without limitation, humans, which compositions comprise of caffeine, theacrine and methyl liberine in either solid or in liquid dosage forms.

According to another embodiment, the total concentration of caffeine, theacrine and methyl liberine in the composition is in the range of 0.01% to 20% by weight based on the total weight of the composition with remaining weight containing majorly of disaccharides derived from enzyme treated sucrose isomers either alone or in combination, provided either in solid or in liquid dosage forms, using suitable carriers or excipients or nutritional adjuvant(s).

The total quantity of methylxanthines was kept in between 0.01 to 20% in enzymatically treated sucrose solution.

In order to achieve the stability, solubility and taste appropriate food grade preservatives, viscosity modifiers and optionally bitterness masking agents like stilbenoids such as resveratrol, pterostilbene were added to the formulation.

The composition of the present invention provides substantial improvement endurance, exercise performance, reduced hunger, and weight loss, and inflammation and pain reduction, prophylactic and curative treatment for neurological disease, learning performance, mood enhancement, alertness, uric acid reduction and management.

The composition of the present invention also provides debittering of methylxanthines due to their ability of the methylxanthines to form complex with glucose moiety of the disaccharides.

The composition further provides slow metabolism of caffeine and low glycimic index due to longer metabolic time required for the breakdown of the treated disaccharide compound compared to untreated sucrose or glucose which results in delayed or extended release of long lasting energy boosters.

The compositions and process for preparation are further described by the following non-limiting examples, which provide the preferred mode of carrying out the process of the present invention. It is to be appreciated that several alterations, modifications, optimizations, alternations of the processes described herein are well within the scope of a person skilled in the art and such alterations, modifications, optimizations, alternations, etc. should be construed to be within the scope of the present inventive concept as is disclosed anywhere in the specification.

### Illustrative Formulations

The formulations listed below are set forth for illustrative purposes only, and should not be used to limit the proper construction of the claims in any manner whatsoever. In the formulations nutraceutical excipients percentage varies from 5-90% w/w.

### Illustrative Formulations are Set Forth Below

TABLE 1
Formulations of Methylxanthines with disaccharides
derived from enzymatic conversion of Sucrose

| S. | Sucrose | Percentage | | Percentage of | Pterostilbene/ | Nutraceutical |
|---|---|---|---|---|---|---|
| NO | Isomer | of Sugar | Methylxanthines | Methylxanthines | Resveratrol | excipients |
| 1 | Turanose | 10-90% | Caffeine | 0.01-20% | 0-20% | 5-90% |
| | | | Theacrine | | | |
| | | | Methylliberine | | | |
| | | | Paraxanthine | | | |
| | | | Theobromine | | | |
| | | | Theophylline | | | |
| 2 | Leucrose | 10-90% | Caffeine | 0.01-20% | 0-20% | 5-90% |
| | | | Theacrine | | | |
| | | | Methylliberine | | | |
| | | | Paraxanthine | | | |
| | | | Theobromine | | | |
| | | | Theophylline | | | |
| 3 | Trehalulose | 10-90% | Caffeine | 0.01-20% | 0-20% | 5-90% |
| | | | Theacrine | | | |
| | | | Methylliberine | | | |
| | | | Paraxanthine | | | |
| | | | Theobromine | | | |
| | | | Theophylline | | | |
| 4 | Isomaltose | 10-90% | Caffeine | 0.01-20% | 0-20% | 5-90% |
| | | | Theacrine | | | |
| | | | Methylliberine | | | |
| | | | Paraxanthine | | | |
| | | | Theobromine | | | |
| | | | Theophylline | | | |

TABLE 2
Formulations of Methylxanthines with disaccharides derived from enzymatic conversion of Sucrose

| | | Percentage | | | | Percentage | | Percentage of |
|---|---|---|---|---|---|---|---|---|
| S. | Sucrose | of Sugar | | Percentage of | | of | Nutraceutical | Nutraceutical |
| NO | Isomer | Isomer | Methylxanthines | Methylxanthines | Stelbenoid | stilbenoid | excipients | excipients |
| 1 | Trehallulose | 12.5 | Caffeine | 0.15 | Pterostilbene | 0 | Calcium | 5 |
| | Isomaltulose | 12.5 | Theacrine | 0.1 | Resveratrol | 0 | stearate | |
| | | | Methylliberine | 0.05 | | | | |
| 2 | Turanose | 12.5 | Caffeine | 0.15 | Pterostilbene | 0 | Calcium | 5 |
| | Lucrose | 12.5 | Theacrine | 0.05 | Resveratrol | 0 | stearate | |
| | | | Methylliberine | 0.1 | | | | |
| 3 | Trehallulose | 25 | Caffeine | 0.15 | Pterostilbene | 0.5 | Calcium | 5 |
| | | | Theacrine | 0.1 | Resveratrol | | stearate | |
| | | | Methylliberine | 0.05 | | | | |
| 4 | Trehallulose | 25 | Caffeine | 0.15 | Pterostilbene | 0.3 | Calcium | 5 |
| | | | Theacrine | 0.1 | Resveratrol | | stearate | |
| | | | Methylliberine | 0.05 | | | | |
| 5 | Turanose | 12.5 | Caffeine | 0.15 | Pterostilbene | | Calcium | 5 |
| | Leucrose | 12.5 | Theacrine | 0.05 | Resveratrol | 0.5 | stearate | |
| | | | Methylliberine | 0.1 | | | | |

### General Procedure for the Preparation of Formulation of Methylxanthines With Sucrose Isomers

To the aqueous solution of enzyme treated sucrose isomers (turanose, leucrose, trehalulose, and isomaltulose or combinations thereof) (90%- 10% w/w) was added, methylxanthine (caffeine, theacrine, methylliberine, paraxanthine, theobromine, theophylline or combinations thereof: 0.01% to 20% w/w), followed by the addition of stilbenoids like resveratrol (0-20% w/w), pterostilbene (0-20% w/w) and nutraceutical excipients like binders, diluents, lubricants, stabilizers, solubilizers, sustained release polymers, suitable coloring agents, taste enhancers, etc (5-90% w/w). The composition was agitated at room temperature to avoid heat-induced inversion of sucrose for 1.0-2.0 h. The resulting solution/suspension was packed for usage.

### Example 1

12.5 g of Trehallusoe and 12.5 g of Isomaltulose were added to 100 ml purified water under stirring at room temperature. Slowly added Caffeine 150 mg, theacrine 100 mg and methylliberine: 50 mg, and allowed to stir for 30 minutes followed by the addition of 5 g of calcium stearate. The resulting solution was stirred for 2-3 h at room temperature.

### Example 2

12.5 g of Turanose and 12.5 g of Lucrose were added to 100 ml purified water under stirring at room temperature. Slowly added Caffeine 150 mg, theacrine 50 mg and methylliberine: 100 mg, and allowed to stir for 30 minutes followed by the addition of 5 g of calcium stearate. The resulting solution was stirred for 2-3 h at room temperature.

### Example 3

25 g of Trehallulose was added to 100 ml purified water under stirring at room temperature. Slowly added Caffeine 150 mg, theacrine 100 mg and methylliberine: 50 mg, and allowed to stir for 30 minutes followed by the addition of 500 mg pterostilbene and 5 g of calcium stearate. The resulting solution was stirred for 2-3 h at room temperature.

### Example 4

25 g of Trehallulose was added to 100 ml purified water under stirring at room temperature. Slowly added Caffeine 150 mg, theacrine 100 mg and methylliberine: 50 mg, and allowed to stir for 30 minutes followed by the addition of 300 mg pterostilbene and 5 g of calcium stearate. The resulting solution was stirred for 2-3 h at room temperature.

### Example 5

12.5 g of Turanose and 12.5 g of Lucrose were added to 100 ml purified water under stirring at room temperature. Slowly added Caffeine 150 mg, theacrine 50 mg and methylliberine: 100 mg, and allowed to stir for 30 minutes followed by the addition of 500 mg resveratrol and 5 g of calcium stearate. The resulting solution was stirred for 2-3 h at room temperature.

Although the invention has been described in detail in the foregoing for the purpose of illustration, it is to be understood that such detail is solely for that purpose and that variations can be made wherein by those skilled in the art without departing from the spirit and scope of the invention except as it may be limited by the claims.

1.-25. (canceled)

26. A composition comprising methylxanthines in combination with disaccharide(s) derived from enzymatic conversion of sucrose, wherein the methylxanthines are in an amount of 0.01% to 20% w/w and disaccharide(s) derived from enzymatic conversion of sucrose are in an amount of 10% to 90% w/w of the composition.

27. The composition as claimed in claim 26, wherein the methylxanthines are either in their pure form or as cocrystals or solvates.

28. The composition as claimed in claim 27, wherein the cocrystals/solvates are prepared using coformers selected from Phenols, Cinnamic Acids, Resveratrol, Pterostilbene, alcohols, or organic acids and mixtures thereof.

29. The composition as claimed in claim 26, wherein the methylxanthines are selected from caffeine, theacrine, methylliberine, paraxanthine, theobromine, theophylline alone or combinations thereof, in an amount of 0.01% to 20% w/w.

30. The composition as claimed in claim 26, wherein the disaccharides derived from enzymatic conversion of sucrose are selected from turanose, leucrose, trehalulose and isomaltulose either alone or combinations thereof, in an amount of 10% to 90% w/w.

31. The composition as claimed in claim 26, wherein the composition further comprises nutraceutical supplement selected from Stilbenoids in an amount of 0.1% to 20% w/w preferably resveratrol or pterostilbene.

32. The composition as claimed in claim 26, wherein the carriers, excipients or nutritional adjuvant(s) are selected from flavoring agents, colorants, viscosity modifiers, preservatives, chelating agents, antioxidants, surface modifiers alone or combinations thereof.

33. The composition as claimed in claim 26 comprising suitable carriers, excipients or nutritional adjuvant(s) in an amount of 0.01% to 20% w/w.

34. The composition as claimed in claim 32, wherein the nutraceutical adjuvant(s) are selected from binders, diluents, lubricants, stabilizers, solubilizers, sustained release polymers, coloring agents, or taste enhancers.

35. The composition as claimed in claim 26, wherein the methylxanthines are selected from caffeine, theacrine, paraxanthine, theobromine, theophylline or methylliberine in an amount of 0.01%- 20% w/w in combination with disaccharides derived from enzyme treated sucrose selected from turanose, leucrose, trehalulose and isomaltulose either alone or in combinations thereof in an amount of 10%- 90% w/w.

36. The composition as claimed in claim 26, wherein the composition is formulated for oral administration in various forms of nutritional supplements, such as powders, tablets, capsules, gel dosage forms or aqueous formulations.

37. A process for the preparation of a composition comprising methylxanthines alone or in combinations thereof along with disaccharides derived from enzyme treated sucrose comprising:
i) adding methylxanthines individually or in combination in an amount of 0.01% to 20% w/w to an aqueous solution of enzymatically treated sucrose in an amount 90 to 10% w/w;
ii) adding stilbenoids in an amount 0 to 20% w/w to the solution of step (i);
iii) adding nutraceutical excipients in an amount 5-90% w/w to the solution of step (ii); and
iv) agitating the solution of step (iii) at room temperature to avoid heat induced inversion of sucrose to avoid heat induced inversion of sucrose to obtain the desired composition.

38. The composition as claimed in claim 26, for use in:
i) stimulating the central nervous system and metabolism;
ii) enhancing energy levels, mood, focus, memory, cognitive function, and alertness;
iii) extending and maintaining locomotor activity, physical performance, and endurance;
iv) increasing fat-burning potential and supporting weight management;
v) reducing symptoms of depression, hunger, sleep deprivation, inflammation, pain, and hyperuricemia;
vi) imparting a hypoglycemic effect; and
vii) the prophylactic and/or therapeutic treatment of neurological disorders, cognitive and learning impairments, and mood disorders.

39. The composition as claimed in claim 26, wherein the composition provides delayed or extended-release delivery.

40. A method of stimulating the central nervous system and metabolism, enhancing energy levels, mood, focus, memory, cognitive function, and alertness, extending and maintaining locomotor activity, physical performance, and endurance, increasing fat-burning potential and supporting weight management, reducing symptoms of depression, hunger, sleep deprivation, inflammation, pain, and hyperuricemia, imparting a hypoglycemic effect, and providing prophylactic and/or therapeutic treatment of neurological disorders, cognitive and learning impairments, and mood disorders in a subject in need thereof, the method comprising administering to the subject a composition comprising methylxanthines in combination with disaccharide(s) derived from enzymatic conversion of sucrose.

41. The composition as claimed in claim 36, for use in:
i) stimulating the central nervous system and metabolism;
ii) enhancing energy levels, mood, focus, memory, cognitive function, and alertness;
iii) extending and maintaining locomotor activity, physical performance, and endurance;
iv) increasing fat-burning potential and supporting weight management;
v) reducing symptoms of depression, hunger, sleep deprivation, inflammation, pain, and hyperuricemia;
vi) imparting a hypoglycemic effect; and
vii) the prophylactic and/or therapeutic treatment of neurological disorders, cognitive and learning impairments, and mood disorders.
